(19) United States
(12) Patent Application Publication
(10) Pub. No.: US 2022/0184349 A1
(43) Pub. Date: Jun. 16, 2022

(54) Guide Wires

(71) Applicant: ASAHI INTECC CO., LTD., Seto-Shi (JP)

(72) Inventors: Kenji YOSHIDA, Seto-shi (JP); Keisuke USHIDA, Seto-shi (JP); Masahiro KASHIWAI, Seto-shi (JP)

(73) Assignee: ASAHI INTECC CO., LTD., Seto-Shi (JP)

(21) Appl. No.: 17/682,052

(22) Filed: Feb. 28, 2022

Related U.S. Application Data
(63) Continuation of application No. PCT/JP2019/033779, filed on Aug. 28, 2019.

(51) Int. Cl. A61M 25/09

(52) U.S. Cl. CPC A61M 25/09; A61M 2025/09083

(57) ABSTRACT

A guide wire includes a core shaft formed of superelastic metal, where the core shaft has a tapered portion having a diameter decreasing from its proximal end toward its distal end, an intermediate portion that is cylindrical and is adjacent to the proximal end of the tapered portion, and a proximal portion adjacent to a proximal end of the intermediate portion and having a maximum diameter larger than a diameter of the intermediate portion; a coil body covering at least a part of the intermediate portion and the tapered portion of the core shaft, the coil body having an outer diameter of 0.36 mm or less; and a distal tip disposed at a distal end of the coil body and forming a distal end of the guide wire, the diameter of the intermediate portion being 0.22 to 0.24 mm.

### CROSS-REFERENCE TO RELATED APPLICATION

This application is a continuation application of International Application No. PCT/JP2019/033779, filed Aug. 28, 2019, the contents of which are incorporated herein by reference in their entirety.

### TECHNICAL FIELD

The disclosed embodiments relate to guide wires.

### BACKGROUND

Known guide wires have been used in the insertion of a catheter and the like into a blood vessel. Among such guide wires, several configurations have been disclosed with a superelastic shaft formed of a nickel-titanium (Ni—Ti) alloy in the vicinity of the guide wire's distal end, such as in Patent Literature 1 (JP2013-544575A) and Patent Literature 2 (JP2005-46603A).

### SUMMARY

### Technical Problem

In guide wires having a shaft formed of nickel-titanium alloy, there has been need for a technology capable of suppressing a delay in torque response.

Note that such problem is not limited to guide wires inserted into the blood vascular system, but is also common in guide wires to be inserted into each of the organs and/or organ systems in the human body such as the lymph gland system, biliary system, urinary system, respiratory tract system, digestive organ system, secretory gland, or genital organs. Moreover, such problem is not limited to a guide wire having a shaft formed of nickel-titanium alloy, but is also common in guide wires having a shaft formed of other superelastic metals.

The disclosed embodiments were made to solve the problem described above, and an objective of the present application is to provide a technology to suppress a delay in torque response in a guide wire.

### Solution to Problem

The disclosed embodiments have been made to solve at least a part of the problem described above, and can be implemented as the following aspects.

(1) One aspect of the disclosed embodiments provides a guide wire. This guide wire includes a core shaft formed of superelastic metal, wherein the core shaft has a tapered portion having a diameter decreasing from its own proximal end toward its distal end, an intermediate portion that is cylindrical and is adjacent to the proximal end of the tapered portion, and a proximal portion adjacent to the proximal end of the intermediate portion and having a maximum diameter larger than a diameter of the intermediate portion; a coil body covering at least a part of the intermediate portion and the tapered portion of the core shaft, the coil body having an outer diameter of 0.36 mm or less; and a distal tip disposed at the distal end of the coil body and forming the distal end of the guide wire, wherein the diameter of the intermediate portion of the core shaft is between 0.22 and 0.24 mm.

This configuration provides an intermediate portion of a core shaft with a relatively thin diameter, 0.22 to 0.24 mm, and thus allows a delay in torque response to be suppressed. In other words, this can provide a guide wire with better rotational performance. Moreover, the guide wire includes a coil body with an outer diameter of 0.36 mm or less that covers at least a part of the intermediate portion and the tapered portion of the core shaft, and thus ensures the guide wire is properly sized even in a thin part of the core shaft. Providing the intermediate portion of the core shaft with a thinner diameter allows the wire diameter of the coil body to be thicker, and thus suppresses occurrences of riding up and collapse of the coil body during use of the guide wire.

(2) In the guide wire of the aspect described above, the coil body may have a constant wire diameter. A configuration in which the wire diameter of a coil body on the proximal end is smaller than the wire diameter of the coil body on the distal end is more likely to cause riding on, collapse, or the like of the coil body in the part of the coil body with a smaller wire diameter when the guide wire is used. By contrast, this configuration is set to have a core shaft with a diameter of the intermediate portion of between 0.22 and 0.24 mm, thereby allowing the wire diameter of the coil body to be relatively thick and of constant size, which suppresses occurrences of riding on, collapse, or the like of the coil body during use of the guide wire.

(3) In the guide wire of the aspect described above, the core shaft may be formed of nickel-titanium (Ni—Ti) alloy. Nickel-titanium alloy has superior restorability, durability, and corrosion resistance, and this configuration can provide a guide wire with superior restorability, durability, and corrosion resistance in the core shaft.

(4) In the guide wire of the aspect described above, the proximal end of the coil body may be fixed to the intermediate portion of the core shaft. This provides a coil body wound around from a predetermined position of the intermediate portion of the core shaft to the distal end of the guide wire, and thus allows increased flexural rigidity of the intermediate portion and a tapered portion, which are relatively thin parts in the guide wire.

(5) In the guide wire of the aspect described above, a proximal end core shaft may be further included and can be connected to the proximal end of the core shaft and formed of a material with higher rigidity than that of the core shaft. This provides a proximal end core shaft with high rigidity, and thus allows improved pushdown and delivery.

(6) In the guide wire of the aspect described above, the proximal end core shaft may be formed of stainless steel. Because of superior formability of stainless steel, this configuration allows easy production of a guide wire with high pushdown and delivery properties and superior rotational performance.

(7) In the guide wire of the aspect described above, a wire rod may be further included and can be connected to the distal end of the core shaft and formed of a material having more plastic deformability than that of the core shaft. The distal tip of the guidewire may be connected between the distal end of the coil body and the distal end of the wire rod. This allows the distal end part of a guide wire to be easily shaped.

Note that the disclosed embodiments can be achieved in various aspects, for example, in a form of a core shaft product or the like formed of a plurality of core shafts used in a guide wire.

The terms “comprise” and any form thereof such as “comprises” and “comprising,” “have” and any form thereof such as “has” and “having,” “include” and any form thereof such as “includes” and “including,” and “contain” and any form thereof such as “contains” and “containing” are open-ended linking verbs. As a result, a device, like a guide wire, that “comprises,” “has,” “includes,” or “contains” one or more elements possesses those one or more elements, but is not limited to possessing only those elements. Likewise, a method that “comprises,” “has,” or “includes” one or more steps possesses those one or more steps, but is not limited to possessing only those one or more steps.

Any embodiment of any of the devices and methods can consist of or consist essentially of—rather than comprise/include/have—any of the described steps, elements, and/or features. Thus, in any of the claims, the term “consisting of” or “consisting essentially of” can be substituted for any of the open-ended linking verbs recited above, in order to change the scope of a given claim from what it would otherwise be using the open-ended linking verb.

### BRIEF DESCRIPTION OF THE DRAWINGS

FIG. 1 is a partial sectional view of a guide wire of a first embodiment.

FIG. 2 is a chart showing a relationship between the diameter of an intermediate portion of a guidewire's core shaft and the guidewire's rotational performance.

FIG. 3 is an illustration of a test for evaluating rotational performance.

FIG. 4 is a graph showing the rotational performance of the guide wire of the first embodiment compared to two other examples.

FIG. 5 is an illustration of the test for evaluating the rotational performance shown in FIG. 4.

FIG. 6 is a partial sectional view of a guide wire of a second embodiment.

FIG. 7 is a partial sectional view of a guide wire of a third embodiment.

FIG. 8 is a partial sectional view of a guide wire of a fourth embodiment.

FIG. 9 is a partial sectional view of a guide wire of a fifth embodiment.

### DETAILED DESCRIPTION

### First Embodiment

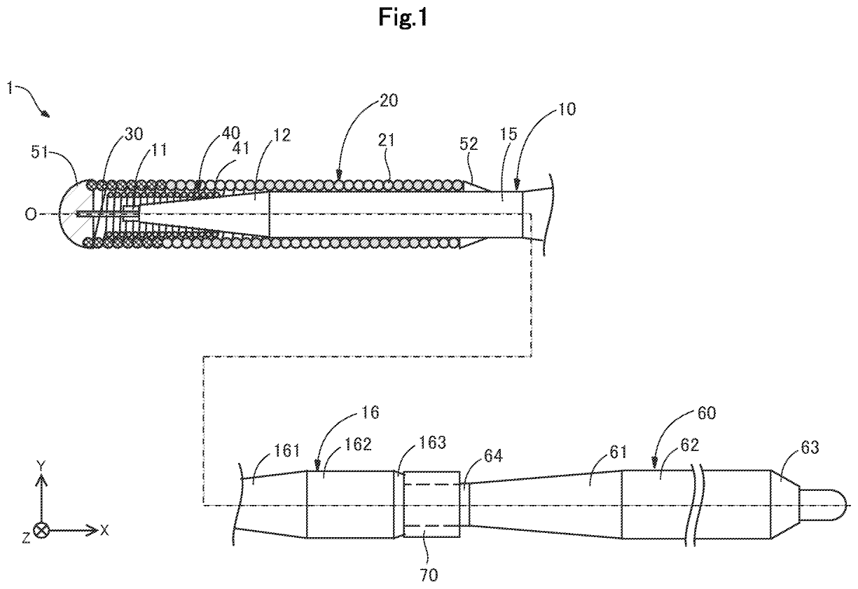

FIG. 1 is a partial sectional view of a guide wire 1 of a first embodiment. The guide wire 1 is a medical appliance used in, e.g., the insertion of a catheter into a blood vessel, and can include a core shaft 10, a coil body 20, a wire rod 30, a coated part 40, a distal tip 51, a proximal end fixing part 52, and a proximal end core shaft 60. FIG. 1 shows an axis O (dash-dot line) passing through the center of the guide wire 1. In the subsequent examples, an axis passing through the center of the core shaft 10 closer to the proximal end than an intermediate portion 15 of the core shaft, an axis passing through the center of the coil body 20, and an axis passing through the center of the coated part 40 coincide with the axis O. However, in other embodiments, the axis passing through the center of the core shaft 10, the axis passing through the center of the coil body 20, and the axis passing through the center of the coated part 40 need not coincide with the axis O.

FIG. 1 illustrates XYZ-axes that are orthogonal to each other. The X-axis corresponds to an axial direction of the guide wire 1, the Y-axis corresponds to a height direction of the guide wire 1, and the Z-axis corresponds to a width direction of the guide wire 1. The left side in FIG. 1 (−X-axis direction) is designated as the “distal end side” of the guide wire 1 and each of its components, and the right side in FIG. 1 (+X-axis direction) is designated as the “proximal end side” of the guide wire 1 and each of its components. Additionally, in the guide wire 1 and each of its components, an end part located at a distal end side is designated as “distal end portion” or simply as “distal end,” and an end part located at a proximal end side is designated as “proximal end portion” or simply as “proximal end.” In the first embodiment, a distal end side corresponds to a “farther side,” and a proximal end side corresponds to “nearer side.” These points are also common to the drawings showing total configurations subsequent to FIG. 1.

The core shaft 10 is a tapered elongated member having a thicker diameter in the vicinity of its proximal end and a thinner diameter in the vicinity of its distal end. The core shaft 10 is formed of nickel-titanium (Ni—Ti) alloy, which is a superelastic metal. The distal end of the core shaft 10 is connected to the wire rod 30, and the proximal end of the core shaft 10 is connected to the proximal end core shaft 60. The core shaft 10 has a small-diameter portion 11, a tapered portion 12, an intermediate portion 15, and a proximal portion 16, in order from the distal end to the proximal end. The length of each part can be determined arbitrarily.

The small-diameter portion 11 of the core shaft 10 is positioned at the distal end of the core shaft 10. The small-diameter portion 11 is a part having the minimum outer diameter in the core shaft 10, and is cylindrical with a constant outline.

The tapered portion 12 is positioned between the small-diameter portion 11 and the intermediate portion 15. The tapered portion 12 is frustoconical and has an outer diameter that decreases from the proximal end of the tapered portion to the distal end of the tapered portion.

The intermediate portion 15 is adjacent to the proximal end of the tapered portion 12, and is positioned between the tapered portion 12 and the proximal portion 16. The intermediate portion 15 is cylindrical and has a constant outer diameter larger than the outer diameter of the small-diameter portion 11. In the first embodiment, the diameter of the intermediate portion 15 is 0.237 to 0.243 mm. As described in detail later, the guide wire 1 of the first embodiment includes a relatively thin-diameter intermediate portion 15, and thus allows delays in torque response to be suppressed.

The proximal portion 16 is adjacent to the proximal end of the intermediate portion 15 and has a maximum diameter larger than the diameter of intermediate portion 15. The proximal end portion 16 includes a first increased-diameter portion 161, a first large-diameter portion 162, and a first reduced-diameter portion 163. The first increased-diameter portion 161 is frustoconical and has a diameter at the distal end of the proximal portion 16 that is identical to the diameter of the intermediate portion 15, wherein the outer diameter of the first increased-diameter portion increases from its distal end to its proximal end. The first large-diameter portion 162 is a part having the maximum outer diameter in the core shaft 10, and is cylindrical and has a constant outer diameter. The first reduced-diameter portion 163 is frustoconical and has a diameter at its distal end that is identical to the diameter of the first large-diameter portion 162, wherein the outer diameter of the first reduced-diameter portion decreases from its distal end to its proximal end.

The outer sides of the small-diameter portion 11, the tapered portion 12, and a part of the intermediate portion 15 that is closer to the distal end of the intermediate portion 15 than to the proximal end of the intermediate portion are covered with the coil body 20 as described later. By contrast, the proximal portion 16 is not covered with the coil body 20, and is exposed from the coil body 20.

The coil body 20 is cylindrical and hollow and is formed by spirally winding a wire 21 onto the core shaft 10 and the wire rod 30. The wire 21 forming the coil body 20 may be a single line composed of one wire, or a strand stranded with a plurality of wires. When the wire 21 is a single line, the coil body 20 is configured as a single coil; and when the wire 21 is a strand, the coil body 20 is configured as a hollow stranded coil. Furthermore, a single coil and a hollow stranded coil may be combined to define the coil body 20. The line diameter of the wire 21 and the mean coil diameter in the coil body 20 (the mean diameter of the outer diameter and the inner diameter of the coil body 20) can be determined arbitrarily. The outer diameter of the coil body 20 is 0.36 mm or less, and the line diameter of the wire 21 (also referred to as wire diameter) is constant.

The proximal end of the coil body 20 is fixed to the intermediate portion 15 of the core shaft 10 with the proximal end fixing part 52. The proximal end fixing part 52 can be formed of any bond, e.g., metal solder such as silver solder, gold solder, zinc, Sn—Ag alloy, or Au—Sn alloy, or adhesive such as an epoxy-based adhesive. The proximal end fixing part 52 can be placed at any position in the intermediate portion 15. It is preferable that the proximal end fixing part 52 be positioned at the proximal portion of the intermediate portion 15, because this allows the coil body 20 to largely cover the intermediate portion 15 and improves flexural rigidity of the intermediate portion 15, which has a relatively thin diameter.

The wire 21 can be formed of, e.g., stainless alloy such as SUS 304, or SUS 316; superelastic alloy such as Ni Ti alloy; a piano wire; radiolucent alloy such as nickel-chromium-based alloy or cobalt alloy; radiopaque alloy such as gold, platinum, tungsten, or alloy containing these elements (e.g., platinum nickel alloy). Note that the wire 21 may be formed of a known material other than those described above. In the first embodiment, the wire 21 is formed of platinum nickel (Pt—Ni) alloy in a first portion covering the wire rod 30, and of stainless alloy in a second portion that is proximal to the first portion. This allows the vicinity of the distal end of the guide wire 1 to be shaped easily. FIG. 1 indicates the wire 21 with cross hatching in a portion formed of platinum nickel (Pt—Ni) alloy, and diagonal hatching in a portion formed of stainless alloy.

The wire rod 30 is an elongated member having a constant outer diameter from the proximal end to the distal end. The cross-sectional shape of the wire rod 30 is elliptical and has a major axis and a minor axis. The wire rod 30 is positioned adjacent to the small-diameter portion 11 of the core shaft 10, with the major axis in the Y-axis direction and the minor axis in the Z-axis direction. The wire rod 30 is formed of a material having more plastic deformability than that of the core shaft 10, e.g., stainless alloy such as SUS 302, SUS 304, or SUS 316. The wire rod 30 is formed of a material having more plastic deformability than that of the core shaft 10, and thus allows the distal end part of the guide wire 1 to be shaped more easily relative to if it lacked the wire rod 30. The wire rod 30 is also referred to as “ribbon.” The proximal end of the wire rod 30 is bonded to the small-diameter portion 11 on the distal end of the core shaft 10 with a bond. Examples of the bonds available include metal solder such as silver solder, gold solder, zinc, Sn—Ag alloy, or Au—Sn alloy, or adhesive such as epoxy-based adhesive. The distal end of the wire rod 30 is fixed to the distal tip 51 as described later.

Note that in the example in FIG. 1, the wire rod 30 is bonded to the core shaft 10, with the position of the proximal end of the wire rod 30 matching the position of the proximal end of the small-diameter portion 11, in the axis O (X-axis) direction. However, in other embodiments there may be difference between the position of the proximal end of the wire rod 30 and the position of the proximal end of the small-diameter portion 11 in the axis O direction. For example, the proximal end of the wire rod 30 may be positioned distally in the −X-axis direction relative to the proximal end of the small-diameter portion 11. Moreover, the wire rod 30 may be connected to the distal end of the small-diameter portion 11, or inserted in and connected to the small-diameter portion 11.

The coated part 40 is a multiple thread coil having a plurality (e.g., 8) of wires 41 wound multiply, and is configured to have less plastic deformability than the wire rod 30 and more plastic deformability than the core shaft 10. The coated part 40 can be formed by, e.g., densely stranding a plurality of the wires 41 so as to contact each other on a cored bar, then removing remaining stress with a known heat treatment method, and extracting the cored bar. The material of the wire 41 may be the same as or different from that of the wire 21. The proximal end of the coated part 40 is bonded to the tapered portion 12 of the core shaft 10 with any bond similar to the proximal end fixing part 52. The coated part 40 reduces the rigidity gap between the core shaft 10 and the wire rod 30, thus suppressing occurrences of local deformation in the vicinity of the joint between the core shaft 10 and the wire rod 30, and preventing breakage of the core shaft 10 and the wire rod 30.

Note that the coated part 40 can employ any aspect as long as it has configuration with less plastic deformability than the wire rod 30 and more plastic deformability than the core shaft 10. For example, the coated part 40 is not limited to a multiple thread coil, and may be a single thread coil formed of one wire, may be a tubular member made of resin, metal, or the like formed tubularly, and may be coated with a hydrophobic resin material, a hydrophilic resin material, or a mixture thereof

The distal tip 51 is positioned at the distal end of the guide wire 1, and holds integrally the distal end of the wire rod 30 and the distal end of the coil body 20. The distal tip 51 can be formed with any bond in the same manner as the proximal end fixing part 52. The distal tip 51 and the proximal end fixing part 52 can employ the same bond or different bonds. The distal tip 51 may be configured to integrally hold the distal end of the wire rod 30, the distal end of the coil body 20, and the distal end of the coated part 40.

The proximal end core shaft 60 is an elongated member formed of stainless steel. The proximal end core shaft 60 includes a joint portion 64, a second increased-diameter portion 61, a second large-diameter portion 62, and a second reduced-diameter portion 63. The joint portion 64 is cylindrical and has a smaller diameter than the diameter of the proximal end of the core shaft 10. The core shaft 10 and the proximal end core shaft 60 are connected via a connection member 70, with the distal end of the joint portion 64 being bonded to the proximal end of the core shaft 10. The connection member 70 is formed of nickel-titanium alloy, also generally referred to as “Ni Ti pipe,” and has superior flexibility, kink prevention, and shape memory. The second increased-diameter portion 61 is frustoconical and has a diameter at its distal end that is identical to the diameter of the joint portion 64, wherein the outer diameter of the second increased-diameter portion increases from its distal end to its proximal end. The second large-diameter portion 62 is a portion having the maximum outer diameter in the proximal end core shaft 60, and is cylindrical and has a constant outer diameter. The second reduced-diameter portion 63 is frustoconical and has a diameter at its distal end that is identical to the diameter of the second large-diameter portion 62, wherein the outer diameter of the reduced-diameter portion decreases from its distal end to its proximal end.

The proximal end core shaft 60 is used when a technician grips the guide wire 1. The proximal end core shaft 60 is formed of stainless steel, thus has higher rigidity than the core shaft 10 formed of Ni—Ti alloy, and allows improved pushdown and delivery of the guide wire 1. Moreover, stainless steel has superior formability, and thus allows easy production of the proximal end core shaft 60. Examples of the stainless steel available include SUS 302, SUS 304, and SUS 316.

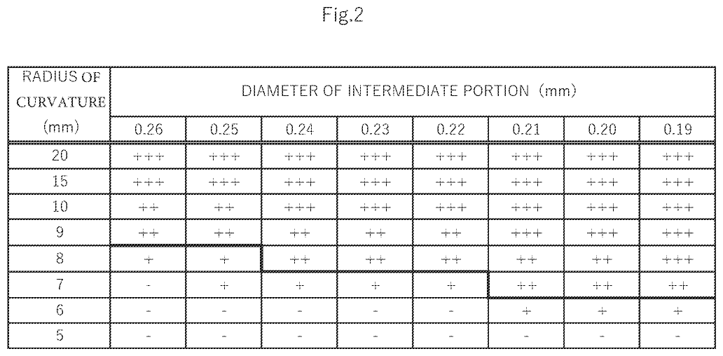

Description will now be provided for the effect of the guide wire 1 of the first embodiment with reference to FIGS. 2-5.

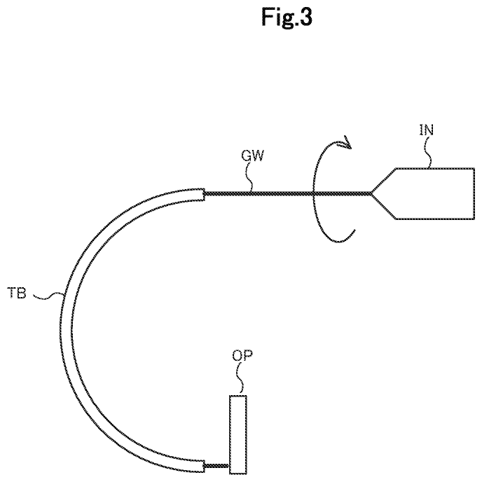

FIG. 2 shows the relationship between the diameter of an intermediate portion of the core shaft and rotational performance of the guide wire. In FIG. 2, the diameter of the intermediate portion is changed to evaluate rotational performance on the basis of the difference between an input angle and an output angle in the below-described test shown in FIG. 3.

In FIG. 2, rotational performance is evaluated for each diameter of the intermediate portions as the radius of curvature shown in FIG. 3 is changed to 5, 6, 7, 8, 9, 10, 15, and 20 mm. In the rotational performance test shown in FIG. 3, the proximal end of a guide wire GW is connected to a rotary device IN, and the distal end of the guide wire GW is connected to a measuring device OP, with the intermediate portion being inserted in and passed through a tube TB formed with a predetermined curvature. The measuring device OP measures an output angle at a distal end of the guide wire GW relative to an input angle as the proximal end of the guide wire GW is rotated at a speed of 1.5 rpm in the rotary device IN. In FIG. 2, a difference between an input angle and an output angle of less than 10° is represented by “+++”; a difference between an input angle and an output angle of 10° or more to less than 50° is represented by “++,” a difference between an input angle and an output angle of 50° or more to less than 100° is represented by “+,” and a difference between an input angle and an output angle of 100° or more is represented by “−.” The difference between the input angle and the output angle recorded in the evaluation is the difference as the change in the output angle relative to the input angle is stable.

In FIG. 2, a bold line indicates evaluation between “++” and “+.” As shown in FIG. 2, a diameter of the intermediate portion of 0.24 mm or less leads to better rotational performance relative to a diameter of the intermediate portion of 0.25 mm or more. Meanwhile, the human blood vessel has a site with a radius of curvature of about 8 mm at a proximal end portion such as a branch of the main trunk and the circumflex of the left coronary artery. In other words, a guide wire may be used in a site having a radius of curvature of about 8 mm. It is thus preferable to use a guide wire with rotational performance evaluated as “++” or “+++” at a radius of curvature of 8 mm. That is, in view of rotational performance, the diameter of the intermediate portion is preferably less than 0.25 mm. By contrast, a core shaft formed of nickel-titanium (Ni—Ti) alloy has less flexural rigidity than a core shaft formed of stainless alloy, and thus preferably has a larger diameter in view of pushdown and delivery. Accordingly, for balancing pushdown and delivery with rotational performance, the diameter of the intermediate portion is preferably 0.20 to 0.24 mm, which has rotational performance evaluated as “++” at a radius of curvature of 8 mm. More preferable is 0.22 to 0.24 mm . The first embodiment employs 0.24 mm, the largest diameter in the range described above.

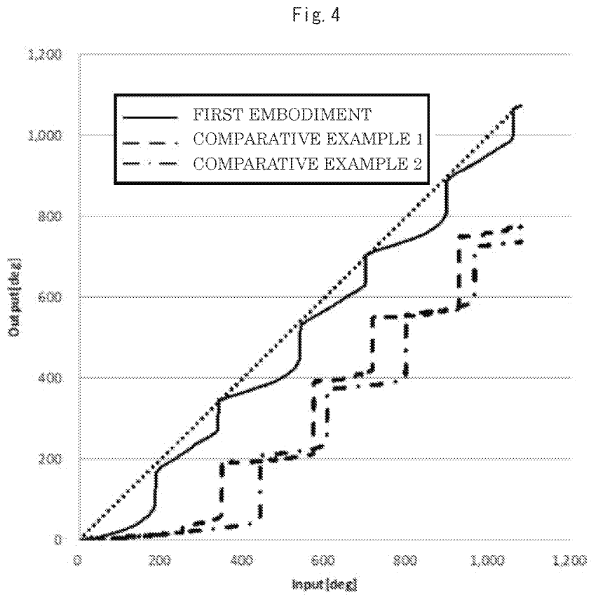

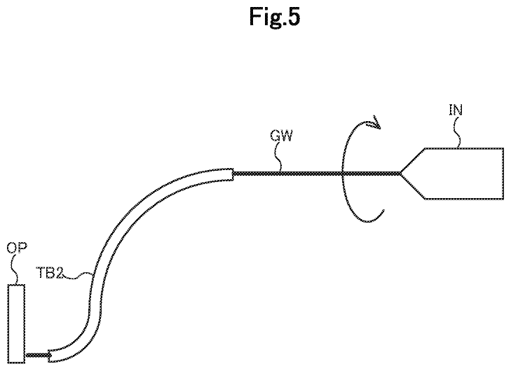

FIG. 4 shows rotational performance of the guide wire 1 of the first embodiment in comparison to that of comparative examples that each have a different diameter in the intermediate portion than the diameter of the intermediate portion of guide wire 1 of the first embodiment. FIG. 5 illustrates the test for evaluating the rotational performance shown in FIG. 4.

The rotational performance test shown in FIG. 5 is a test similar to the test shown in FIG. 3, but the tube TB2 used in the FIG. 5 test is different from the tube TB shown in FIG. 3 and has a two-step curvature. In the tube TB2, the portion of the tube close to the rotary device IN has a radius of curvature of 70 mm, and the portion of the tube close to the measuring device OP has a radius of curvature of 3 mm. FIG. 4 is a graph showing the output angle against the input angle as the proximal end of the guide wire GW is rotated at a speed of 1.5 rpm in the rotary device IN. FIG. 4 depicts the results for the guide wire 1 of the first embodiment with a solid line, Comparative Example 1 with a dashed line, and Comparative Example 2 with a dash-dot line. Comparative Examples 1 and 2 are third-party products, each of which has an intermediate portion with a diameter of 0.25 mm in a guide wire. FIG. 4 indicates ideal behavior with no delay of torque response with a dotted line. As shown in FIG. 4, the guide wire 1 of the embodiment exhibits a slight delay in torque response, but displays nearly ideal behavior. In other words, the guide wire 1 of the first embodiment suppresses a delay in torque response relative to the guide wires of the comparative examples.

As described above, the guide wire 1 of the first embodiment has an intermediate portion 15 of a core shaft 10 with a relatively thin diameter, 0.24 mm, and thus allows suppression of a delay in torque response. In other words, a guide wire with good rotational performance can be provided.

Moreover, the guide wire 1 of the first embodiment includes the coil body 20 with an outer diameter of 0.36 mm or less that covers a part of the intermediate portion 15 and the tapered portion 12 of the core shaft 10, and thus ensures a proper size of the guide wire 1 even in a thin part of the core shaft 10.

Furthermore, in the guide wire 1 of the first embodiment, the coil body 20 has a constant wire diameter (mean diameter of the wire 21). For example, when an intermediate portion having a larger diameter than the diameter of the first embodiment's intermediate portion 15 is used instead of the intermediate portion 15 of the guide wire 1 of the first embodiment, a coil body is set to have a smaller wire diameter in a part covering the intermediate portion than the wire diameter in a part covering the tapered portion in order to keep a constant outer diameter of the coil body. Use of a guide wire employing such coil body may result in riding on, collapse, or the like of the coil body in the part of the coil body having a smaller wire diameter during use of the guide wire. By contrast, the guide wire 1 of the first embodiment allows for a relatively thick, constant wire diameter of the coil body 20 by setting the diameter of the intermediate portion 15 to 0.24 mm (to be thinner), and thus suppresses occurrences of riding on, collapse, or the like of the coil body 20 during use of the guide wire 1.

Additionally, the guide wire 1 of the first embodiment employs a core shaft made of nickel-titanium alloy in the vicinity of the distal end side. Nickel-titanium alloy is a superelastic metal and thus can provide a guide wire with restorability. Particularly, since nickel-titanium alloy has superior restorability, durability, and corrosion resistance among superelastic metals, the guide wire 1 of the embodiment enables improving restorability, durability, and corrosion resistance of the core shaft 10.

Moreover, the guide wire 1 of the first embodiment has the coil body 20 wound around from a predetermined position of the intermediate portion 15 of the core shaft 10 to the distal end of the guide wire 1, and thus allows increased flexural rigidity of the intermediate portion 15 and the tapered portion 12, which are relatively thinner parts in the guide wire 1.

Furthermore, the guide wire 1 of the first embodiment includes the proximal end core shaft 60 formed of stainless steel causing higher rigidity of the proximal end core shaft 60 than that of the core shaft 10, and thus allows improved pushdown and delivery. Consequently, it is possible to provide the guide wire 1 having high pushdown and delivery, and superior rotational performance.

As described above, the core shaft 10 is formed of a superelastic material, and the wire rod 30 is formed of a material having more plastic deformability than that of the core shaft 10. The coated part 40 is configured to have less plastic deformability than the wire rod 30 and more plastic deformability than the core shaft 10. The coated part 40 reduces the rigidity gap between the core shaft 10 and the wire rod 30 which have different rigidities, and thus allows shaping onto a joint portion between the core shaft 10 and the wire rod 30 to be easier relative to a configuration without the coated part 40. Moreover, reducing the rigidity gap between the core shaft 10 and the wire rod 30 in the coated part 40 protects a locally deformable part generated in the vicinity of the joint portion between the core shaft 10 and the wire rod 30, and suppresses breakage of the core shaft 10 and the wire rod 30. Consequently, it is possible to improve durability of the guide wire 1.

Additionally, in the guide wire 1 of the first embodiment, the distal end of the coated part 40 is located distally of the proximal end of the wire rod 30 and coats a half or more of the wire rod 30 (FIG. 1). In other words, in the guide wire 1 of the embodiment, the coated part 40 is positioned up to the vicinity of the distal tip 51. In this manner, protection is made for a major part of the wire rod 30, which is formed a material having high plastic deformability, thereby suppressing breakage of the wire rod 30 in association with shaping and use, and providing more improved durability of the guide wire 1.

### Second Embodiment

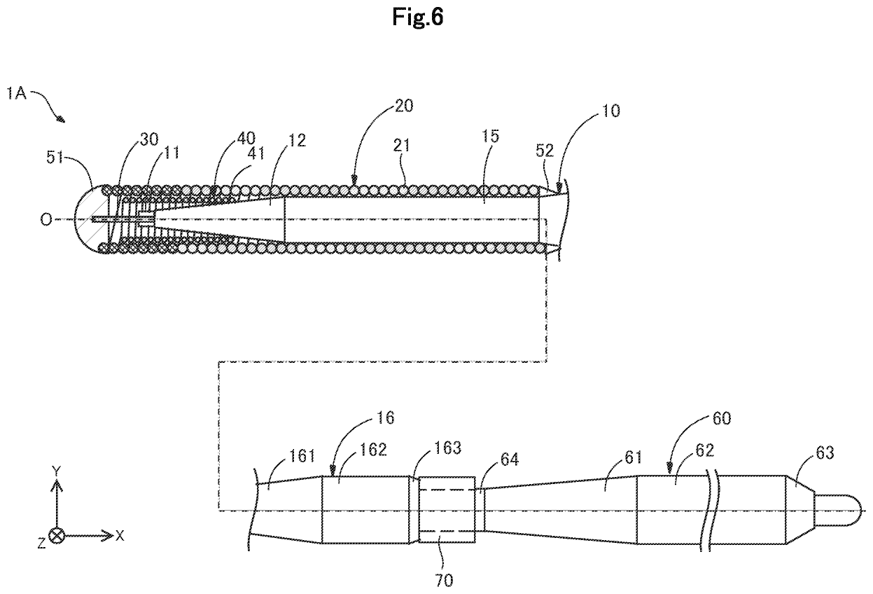

FIG. 6 is a partial sectional view of a guide wire 1A of a second embodiment. The guide wire 1A in the second embodiment includes the proximal end fixing part 52 on the first increased-diameter portion 161 of the proximal end portion 16. In other words, the proximal end of the coil body 20 is not fixed to the intermediate portion 15 of the core shaft 10, and is fixed to the proximal portion 16 of the core shaft 10.

The guide wire 1A of the second embodiment has a coil body 20 wound around from the proximal end of the intermediate portion 15 of the core shaft 10 to the distal end of the guide wire 1A, and thus increases the flexural rigidity of the whole of the intermediate portion 15 and the tapered portion 12, which are relatively thin parts in the guide wire 1A.

### Third Embodiment

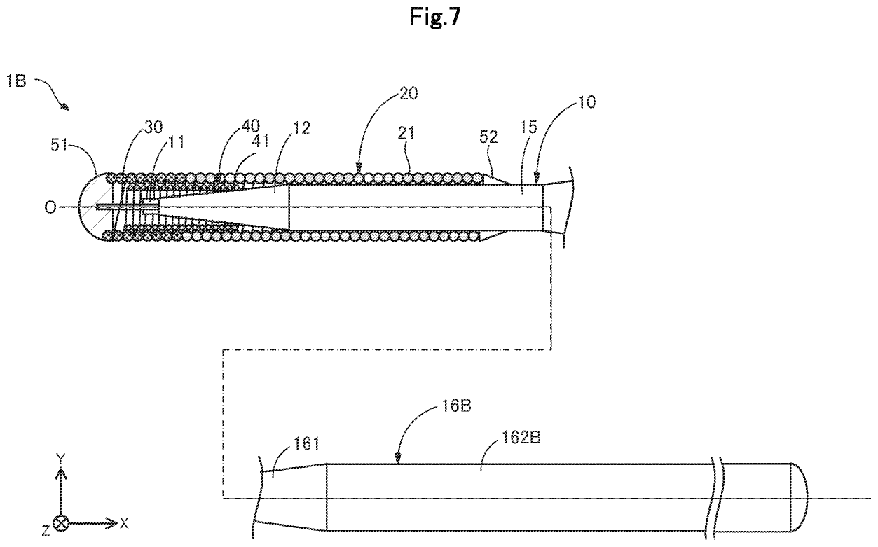

FIG. 7 is a partial sectional view showing a guide wire 1B of a third embodiment. The guide wire 1B of the third embodiment does not include the proximal end core shaft 60. In other words, a proximal portion 16B formed of nickel-titanium alloy is positioned at the proximal end of the guide wire 1B of the embodiment. The proximal portion 16B in the guide wire 1B of the embodiment has a first large-diameter portion 162B with a longer length relative to the proximal end portion 16 in the guide wire 1 of the first embodiment, and does not include the first reduced-diameter portion 163.

The guide wire 1B of the third embodiment does not include the proximal end core shaft 60 made of stainless steel, but has the first large-diameter portion 162B, which has the largest diameter in the core shaft 10, placed at the proximal end of the guide wire 1B, and thus ensures pushout and delivery are enabled. In other words, the third embodiment can also provide the guide wire 1B, which has good pushdown and delivery, and rotational performance.

### Fourth Embodiment

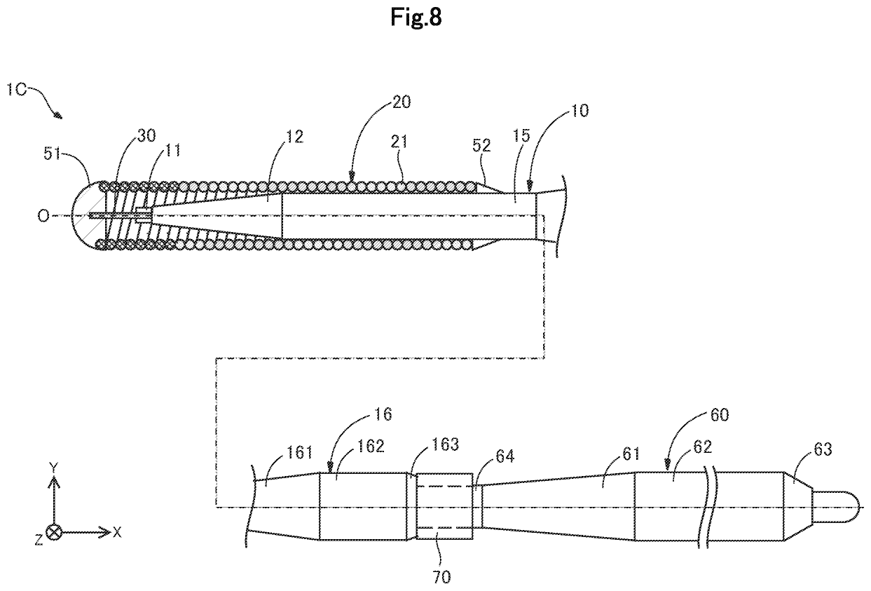

FIG. 8 is a partial sectional view showing a guide wire 1C of a fourth embodiment. The guide wire 1C of the fourth embodiment does not include the coated part 40. Nevertheless, it includes the coil body 20, and thus ensures flexural rigidity in the vicinity of the distal end of the guide wire 1C. Even the guide wire 1C of the fourth embodiment can provide an effect similar to the first embodiment.

### Fifth Embodiment

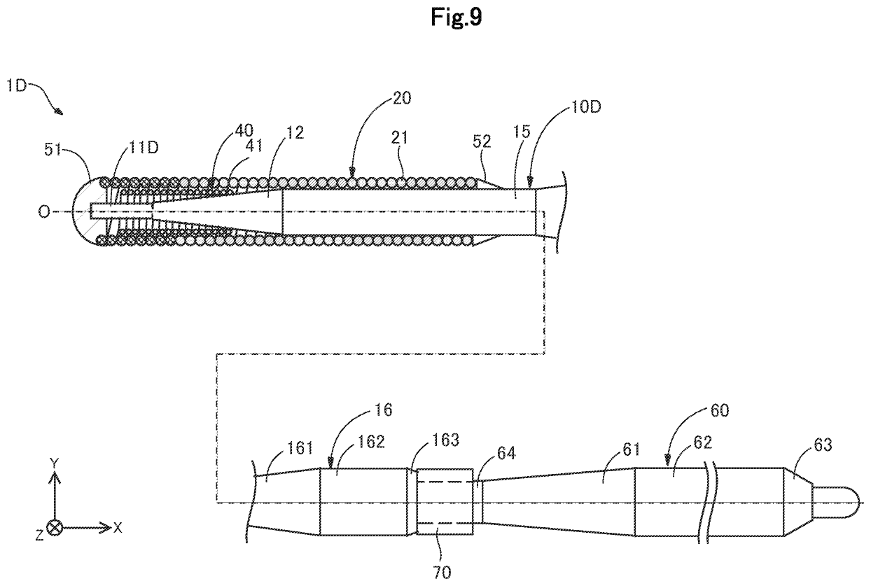

FIG. 9 is a partial sectional view of a guide wire 1D of a fifth embodiment. The guide wire 1D of the fifth embodiment does not include the wire rod 30. In other words, as illustrated, a small-diameter portion 11D of the core shaft 10D extends up to the distal tip 51. In addition, the distal tip 51 integrally holds the distal end of the core shaft 10D and the distal end of the coil body 20. Even with this, the intermediate portion 15 of the core shaft 10D has a relatively thin diameter, 0.24 mm, and it thus can suppress a delay in torque response.

### Variants of the Embodiment

The disclosed embodiments are not limited to the embodiments described above, and can be performed in various aspects in the range without departing from the spirit of the invention, and, for example, the following variants are available.
The guide wire of each of the embodiments described above has been described as a medical appliance used in the insertion of a catheter into a blood vessel, but it can also be configured as a guide wire to be inserted into each of the organs and/or organ systems in the human body such as the lymph gland system, biliary system, urinary system, respiratory tract system, digestive organ system, secretory gland, or genital organs.
In each of the embodiments described above, the intermediate portion 15 is cylindrical with a constant diameter as an example, but the shape of the intermediate portion is not limited to that employed in the embodiments described above. For example, the shape of intermediate portion 15 may qualify as cylindrical as used herein with a diameter that changes continuously or intermittently but remains substantially constant. When the diameter of the intermediate portion 15 is changed in this manner, the mean diameter may be 0.22 to 0.24 mm, or the diameter may be changed while remaining in the range of 0.22 to 0.24 mm.
In the embodiments described above, the core shaft 10 formed of nickel-titanium (Ni—Ti) alloy has been exemplified, but the material forming the core shaft 10 is not limited to that employed the embodiments described above. Various superelastic metals can be used such as a Ni—Ti-based alloy, which is an alloy of Ni—Ti and another metal such as Cu, and a Cu-based alloy such as Cu—Zn—Al alloy.
The configuration of the core shaft is not limited to the embodiments described above. For example, the core shaft 10 of the embodiments described above may not include the small-diameter portion 11. When the core shaft does not include a small-diameter portion, the wire rod 30 can be bonded to a tapered portion of the core shaft. Moreover, in each of the embodiments, the core shaft may comprise a plurality of core shaft members bonded together. In this case, each of the core shaft members may be formed of the same material, or may be formed of different superelastic metals.
In the embodiments described above, the proximal end core shaft 60 formed of stainless steel has been exemplified, but the proximal end core shaft 60 can be formed of various materials having higher rigidity than that of the core shaft 10. For example, a high rigidity material can be used such as chromium-molybdenum steel, nickel-chromium-molybdenum steel, Inconel (Inconel as a trademark), or Incoloy (Incoloy as a trademark).
The cross-sectional shape of each of the parts bonding the small-diameter portion 11 of the core shaft 10 to the wire rod 30 is not limited to that employed the embodiments described above. For example, various cross-sectional shapes can be employed such as a circular shape, a polygonal shape, or a shape having a groove in a circle, ellipse, or the like.
In the embodiments described above, the coil body 20 having a constant wire diameter has been exemplified, but the wire diameter of the coil body 20 may not be constant. For example, the wire diameter of a part of the coil body 20 covering the tapered portion 12 of the core shaft 10, the small-diameter portion 11, and the wire rod 30 may be larger than the wire diameter of a part of the coil body 20 covering the intermediate portion 15.
The configuration of the coil body is not limited to the embodiment described above. For example, the coil body may be configured to be densely wound with no space between adjacent wires, or may be sparsely wound with a space between adjacent wires, or may have a configuration with a mixture of dense winding and sparse winding. In addition, the coil body may include a resin layer coated with, e.g., a hydrophobic resin material, a hydrophilic resin material, or a mixture thereof. As another example, the cross-sectional shape of the wire of the coil body may not be circular.
In the embodiments described above, an example has been shown where the coil body 20 is formed of platinum nickel (Pt—Ni) alloy in the vicinity of the distal end, and of stainless alloy in a portion closer to the proximal end, but the coil body may be wholly formed of the same material. Additionally, formation may be made by, e.g., using three or more different materials and changing a material along an axial direction.

The aspects have been described on the basis of embodiments and variants so far, but the embodiments of the aspects described above are provided to facilitate understanding the aspects and not to limit the aspects. The aspects can be altered or modified without departing from the spirit and scope of the invention, and encompass the equivalents thereof. In addition, the technical features can be appropriately deleted as long as it has not been described as an essential feature herein.

1. A guide wire, comprising:
a core shaft formed of superelastic metal, wherein the core shaft has:
a tapered portion having a diameter decreasing from a proximal end of the tapered portion toward a distal end of the tapered portion;
an intermediate portion that is cylindrical and is adjacent to the proximal end of the tapered portion; and
a proximal portion adjacent to the proximal end of the intermediate portion and having a maximum diameter larger than a diameter of the intermediate portion;
a coil body covering at least a part of the intermediate portion and the tapered portion of the core shaft, the coil body having an outer diameter of 0.36 mm or less; and
a distal tip disposed at a distal end of the coil body and forming a distal end of the guide wire;
wherein the diameter of the intermediate portion of the core shaft is 0.22 to 0.24 mm.

2. The guide wire according to claim 1, wherein the coil body has a constant wire diameter.

3. The guide wire according to claim 1, wherein the core shaft is formed of nickel-titanium (Ni—Ti) alloy.

4. The guide wire according to claim 1, wherein a proximal end of the coil body is fixed to the intermediate portion of the core shaft.

5. The guide wire according to claim 1, further comprising a proximal end core shaft connected to a proximal end of the core shaft and formed of a material with higher rigidity than a material of the core shaft.

6. The guide wire according to claim 5, wherein the proximal end core shaft is formed of stainless steel. The guide wire according to claim 1, further including a wire rod connected to a distal end of the core shaft and formed of a material having more plastic deformability than a material of the core shaft, wherein the distal tip is connected between the distal end of the coil body and the distal end of the wire rod.

8. The guide wire according to claim 2, wherein the core shaft is formed of nickel-titanium (Ni—Ti) alloy.

9. The guide wire according to claim 2, wherein a proximal end of the coil body is fixed to the intermediate portion of the core shaft.

10. The guide wire according to claim 2, further comprising a proximal end core shaft connected to a proximal end of the core shaft and formed of a material with higher rigidity than a material of the core shaft.

11. The guide wire according to claim 2, further including a wire rod connected to a distal end of the core shaft and formed of a material having more plastic deformability than a material of the core shaft, wherein the distal tip is connected between the distal end of the coil body and the distal end of the wire rod.

12. The guide wire according to claim 3, wherein a proximal end of the coil body is fixed to the intermediate portion of the core shaft.

13. The guide wire according to claim 3, further comprising a proximal end core shaft connected to a proximal end of the core shaft and formed of a material with higher rigidity than a material of the core shaft.

14. The guide wire according to claim 3, further including a wire rod connected to a distal end of the core shaft and formed of a material having more plastic deformability than a material of the core shaft, wherein the distal tip is connected between the distal end of the coil body and the distal end of the wire rod.

15. The guide wire according to claim 4, further comprising a proximal end core shaft connected to a proximal end of the core shaft and formed of a material with higher rigidity than a material of the core shaft.

16. The guide wire according to claim 4, further including a wire rod connected to a distal end of the core shaft and formed of a material having more plastic deformability than a material of the core shaft, wherein the distal tip is connected between the distal end of the coil body and the distal end of the wire rod.

17. The guide wire according to claim 5, further including a wire rod connected to a distal end of the core shaft and formed of a material having more plastic deformability than a material of the core shaft, wherein the distal tip is connected between the distal end of the coil body and the distal end of the wire rod.

18. The guide wire according to claim 6, further including a wire rod connected to a distal end of the core shaft and formed of a material having more plastic deformability than a material of the core shaft, wherein the distal tip is connected between the distal end of the coil body and the distal end of the wire rod.
